(19) United States
(12) Patent Application Publication
(10) Pub. No.: US 2010/0186738 A1
(43) Pub. Date: Jul. 29, 2010

(54) LIQUID CARTRIDGE

(75) Inventors: Masaya Kobayashi, Yokohama-shi (JP); Haruhiko Terai, Yokohama-shi (JP); Hiroyuki Wada, Machida-shi (JP); Kenichi Sekine, Ageo-shi (JP); Taisuke Iwashita, Honjo-shi (JP); Yoshihisa Negishi, Oosato-gun (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/668,892

(22) PCT Filed: Jul. 16, 2008
(86) PCT No.: PCT/JP2008/063217

§ 371 Date: Jan. 13, 2010

(30) Foreign Application Priority Data
Jul. 26, 2007 (JP) 2007-193996
Jun. 24, 2008 (JP) 2008-163977

(51) Int. Cl. A61M 11/00; A61M 15/00

(52) U.S. Cl. 128/200.14

(57) ABSTRACT

A liquid cartridge of the present invention has a liquid ejection portion 1 for ejecting liquid droplets, a reservoir E1 for storing liquid, and a communicating member 3 for providing communication between both of them. The reservoir E1 has first and second storage portions 10a, 10b isolated by films 6 to 8 arranged spaced from each other and an atmospheric release portion. When a small diameter portion 5a is inserted into an applied concave portion 4 of the liquid ejection portion 1, a tip 3a of the communicating member 3 breaks through the first to third films 6 to 8 sequentially in the period from the beginning to the end of insertion. Consequently, liquid in the first storage portion 10a is filled into the ejection head 2 and thereafter liquid stored in the second storage portion 10b is filled.

### TECHNICAL FIELD

The present invention relates to a liquid cartridge used mainly for an inhaler which ejects liquid such as medicine, aroma chemical and nicotine into the inhalation flow path of the inhaler in minute liquid droplets for inhalation by a user.

### BACKGROUND ART

A suction unit is generally provided to fill ink from an ink reservoir to an ejection head and to perform restoration operation in an ink jet recording apparatus (see Japanese Patent Application Laid-Open No. 2001-246759). On the other hand, there are also techniques for filling and restoration not using a suction unit as in the ink jet recording apparatus for the purpose of preventing increase in the size of the apparatus in an inhaler which ejects treatment medicine for inhalation by a user using the principle of the ink-jet type ejection. For example, Japanese Patent Application Laid-Open No. 2004-290593 discloses a method of filling liquid into the ejection head by pressing part of a container storing the liquid when the container is attached to an attachment portion of an inhaler. In addition, Japanese Patent Application Laid-Open No. 2004-97617 discloses a restoration unit for an ejection head in which a small amount of liquid is periodically ejected (preliminary ejection) from a liquid droplet ejection portion of an inhaler thereby to eliminate abnormal ejection.

When liquid is filled into the ejection head at the time of first use, filling operation needs to be performed until some of the liquid overflows from the ejection port in order to reliably fill the liquid into the ejection head. In addition, in the case of periodically performing preliminary ejection, liquid of the same kind as used for inhalation is used. In the above-mentioned conventional arts, indispensable liquid, which is the liquid to be inhaled, is ejected uselessly. There is also a problem that the configuration of the restoration unit is complex and it is difficult to improve the portability by reduction of the weight.

### DISCLOSURE OF THE INVENTION

The present invention has been made in view of the above-mentioned problems of conventional arts, and has an object to provide a liquid cartridge which has improved portability and which does not uselessly eject the indispensable liquid to be inhaled.

In order to achieve the above object, a liquid cartridge of the present invention is a liquid cartridge used mainly for an inhaler of liquid, characterized in that the liquid cartridge has:

a reservoir having first and second storage portions isolated by isolation films;

a liquid ejection portion having an ejection head for ejecting liquid droplets and an applied portion to which the reservoir is applied; and

a communicating member for sequentially providing communication of the first and the second storage portions with the ejection head in a period from a beginning to an end of operation of applying the reservoir to the applied portion; wherein

the second storage portion stores indispensable liquid and the first storage portion stores liquid of a kind other than the indispensable liquid.

The liquid cartridge of the present invention is configured as described above and therefore has advantages as described below.

In restoration operation of an ejection head, liquid of a kind other than the indispensable liquid (main inhalation liquid) to be inhaled by the user can be ejected. Therefore, the precious main inhalation liquid is not ejected uselessly. In addition, since a restoration unit of a complex configuration may not be provided, the weight can be reduced.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIGS. 1A and 1B illustrate a liquid cartridge for inhalation according to Embodiment 1, where FIG. 1A is a schematic sectional view of the reservoir and the ejection head before communication, and FIG. 1B is a schematic sectional view after communication.

FIGS. 2A and 2B are views describing the manufacturing process of the reservoir in a liquid cartridge for inhalation of the present invention.

FIGS. 3A and 3B illustrate a liquid cartridge for inhalation according to Embodiment 2, where FIG. 3A is a schematic sectional view of the reservoir and the ejection head before communication, and FIG. 3B is a schematic sectional view after communication.

FIGS. 4A and 4B illustrate a liquid cartridge for inhalation according to Embodiment 3, where FIG. 4A is a schematic sectional view of the reservoir and the ejection head before communication, and FIG. 4B is a schematic sectional view after communication.

FIGS. 5A and 5B illustrate a liquid cartridge for inhalation according to Embodiment 4, where FIG. 5A is a schematic sectional view of the reservoir and the ejection head before communication, and FIG. 5B is a schematic sectional view after communication.

FIG. 6 is a schematic perspective view illustrating an example in which a liquid cartridge of the present invention is applied to an inhaler.

### BEST MODES FOR CARRYING OUT THE INVENTION

Preferred embodiments of the present invention will now be described in detail in accordance with the accompanying drawings.

The liquid cartridge of the present invention is attachable to a liquid ejection device such as an inhaler and is used to eject medicine inhaled by the user. However, not limited to an inhaler, application can be made to atomizing devices in general. Other applications such as aroma chemical generators and transcutaneous administration apparatus are conceivable.

In the present invention, “indispensable liquid” refers to medicine to be administered to the user in the case of use in one of an inhaler and a transcutaneous administration apparatus, and refers to liquid including the main component of fragrance in the case of an aroma chemical generator: in other words, liquid for the main purpose of the liquid cartridge. In the present invention, “liquid of an other kind” is liquid of a kind other than the above indispensable liquid: in the case of use in one of an inhaler and a transcutaneous administration apparatus, liquid for filling to the ejection head. In this case, liquid of an other kind is used for preliminary ejection not inhaled by the user, for example.

In the case of use in an inhaler, the “liquid cartridge” includes a refill container storing the medicine to be inhaled. The liquid cartridge of the present invention is configured to be attachable to an inhaler, and an ejection head for ejecting liquid can be integrated therewith. In the following, description will be made assuming the case of attachment to an inhaler.

### Embodiment 1

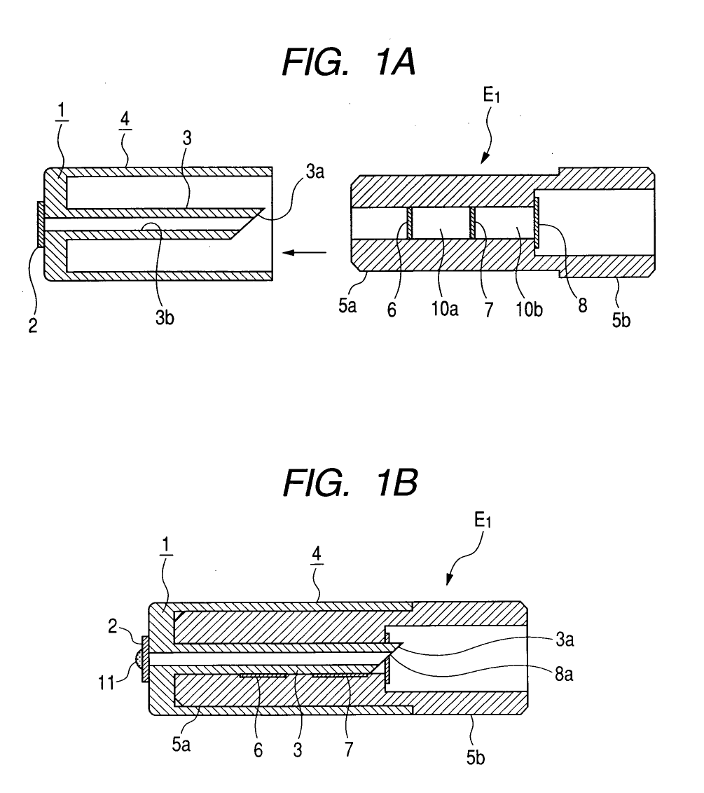

FIGS. 1A and 1B illustrate a liquid cartridge according to Embodiment 1. FIG. 1A is a schematic sectional view before communication is provided between the reservoir and the ejection head. FIG. 1B is a schematic sectional view after communication is provided between the reservoir and the ejection head.

As shown in FIG. 1A, the liquid cartridge has a liquid ejection portion 1 for ejecting liquid in liquid droplets, a reservoir E1 for storing liquid and a communicating member 3 for providing communication between the liquid ejection portion 1 and the storage portions of the reservoir E1 in use. The reservoir E1, having a main body of a cylindrical shape, has a small diameter portion 5a of small outer and inner diameters and a large diameter portion 5b of slightly larger outer and inner diameters than those of the small diameter portion 5a. The small diameter portion 5a has therein a first storage portion 10a and a second storage portion 10b, isolated by films 6, 7, 8 which are three isolation films arranged spaced from one another. The isolation film is not limited to a film but may also be a thing such as a thin plate which functions to separate the storage portions.

The first storage portion 10a stores therein liquid of a kind other than indispensable liquid to be inhaled, and the second storage portion 10b stores therein the indispensable liquid to be inhaled.

The liquid ejection portion 1 has an ejection head 2 for ejecting liquid droplets by means of ejection energy generated by ejection energy generating devices such as an electrothermal transducer (heater) and an electropressure transducer (piezoelectric element). The liquid ejection portion 1 also has an applied concave portion 4 which is an applied portion to which the small diameter portion 5a of the reservoir E1 is detachably fitted, and the communicating member 3 arranged concentrically with the applied concave portion 4.

The first storage portion 10a and second storage portion 10b of the reservoir E1 and the liquid ejection portion 1 are not communicating before use of the inhaler. The communication is sequentially provided by insertion of the reservoir E1 into the applied concave portion 4 of the liquid ejection portion 1 upon use of the inhaler. That is, the communicating member 3 is in the form of a needle, the outer diameter of which is designed to be substantially equal to the inner diameter of the small diameter portion 5a of the reservoir E1. In addition, the communicating member 3 has a liquid flow path 3b provided therein which forms a space for communication between the storage portions 10a, 10b and the ejection head 2 to guide liquid in the storage portions 10a, 10b to the ejection head.

To start inhalation of liquid by means of the inhaler, attachment is performed by inserting the small diameter portion 5a of the reservoir E1 into the applied concave portion 4 provided in the liquid ejection portion 1 in the direction of an arrow shown in the figure, as shown in FIG. 1A. Liquid is filled into the ejection head 2 in the period from the beginning to the end of this attachment operation. This effect is due to the fact that volume of the liquid flow path 3b is smaller than volume of the small diameter portion 5a by the amount of space occupied by the communicating member 3 itself.

In the present embodiment, a pointed tip 3a of the communicating member 3 breaks through the first film 6 to make the first storage portion 10a communicate with the ejection head 2 so that liquid of a kind other than the indispensable liquid is supplied to the ejection head 2. Next, the tip 3a of the communicating member 3 breaks through the second film 7 to make the second storage portion 10b communicate with the ejection head 2 so that the indispensable liquid is supplied. In other words, a communicating member 3 is provided which makes the first and second storage portions 10a, 10b communicate sequentially with the ejection head 2.

Thereafter, as shown in FIG. 1B, the tip portion 3a of the communicating member 3 breaks through the third film 8 and opens into the large diameter portion 5b to be released to the atmosphere; and a small amount of liquid 11 is caused to overflow from the ejection port of the ejection head 2 to complete preparation for the next ejection. The liquid 11 overflowing from the ejection port upon completion of filling liquid into the ejection head is a liquid not used for inhalation (liquid of an other kind). Therefore, the medicine used for inhalation is not wasted. Especial effectiveness is obtained in the case of an expensive medicine used. In addition, a user sometimes performs before inhalation a small amount of preliminary ejection which is not inhaled. Also in this case, according to the present embodiment, liquid not used for inhalation can be ejected.

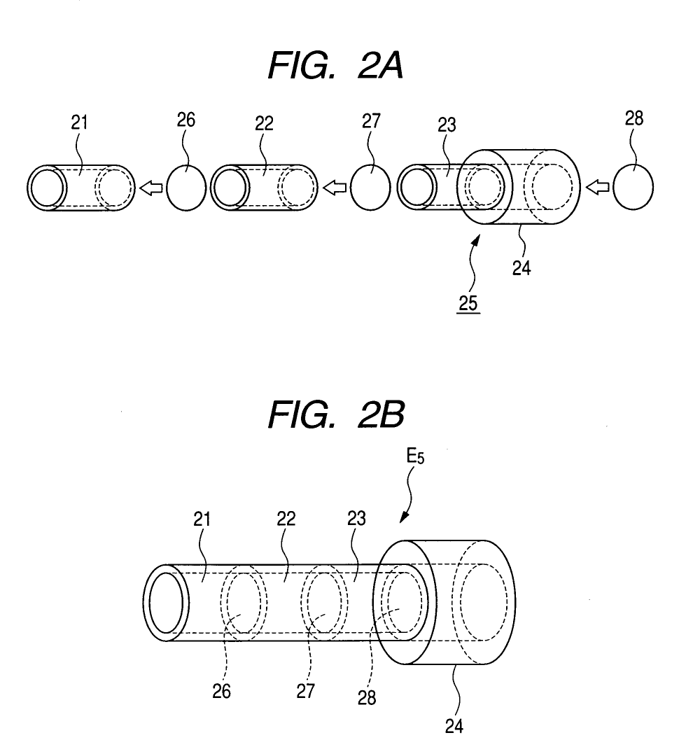

FIGS. 2A and 2B are views describing the manufacturing process of a reservoir E₅. As shown in FIG. 2A, a film 26 is bonded or welded to one end face of a first division small diameter portion 21 to seal the opening. Next a film 27 is bonded or welded to one end face of a second division small diameter portion 22 to seal the opening, and thereafter liquid of a kind other than the indispensable liquid is filled. Next the film 26 on the one end face of the first division small diameter portion 21 is bonded or welded to the other end face of the second division small diameter portion 22 thereby to perform sealing.

Next, a film 28 is bonded or welded to a step portion between a division small diameter portion 23 and a large diameter portion 24 of a division reservoir having the large diameter portion 24 integrally provided on one end of the third division small diameter portion 23, thereby to perform sealing. Thereafter, liquid to be inhaled is filled.

### Embodiment 2

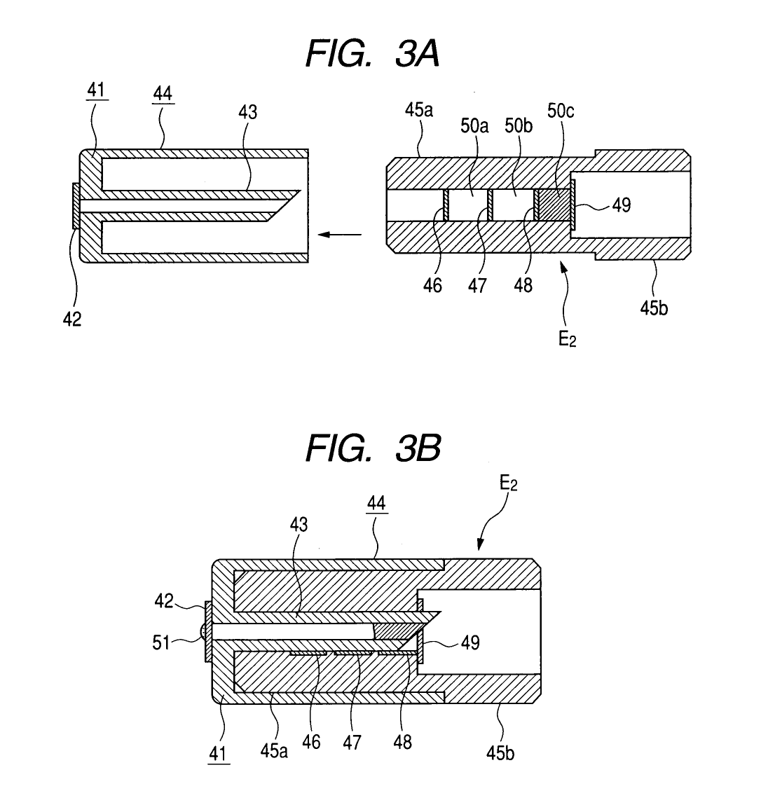

FIGS. 3A and 3B illustrate a liquid cartridge according to Embodiment 2. FIG. 3A is a schematic sectional view of the reservoir and the ejection head before communication. FIG. 3B is a schematic sectional view of the reservoir and the ejection head after communication.

As shown in FIGS. 3A and 3B, a reservoir E2 is a cylindrical body having a small diameter portion 45a and a large diameter portion 45b of outer and inner diameters slightly larger than those of the small diameter portion 45a. The small diameter portion 45a has first to third storage portions 50a to 50c provided therein which are isolated by four films 46 to 49 arranged spaced from one another.

The second storage portion 50b stores indispensable liquid to be inhaled, the first storage portion 50a stores liquid of a kind other than the indispensable liquid, and the third storage portion 50c stores solids.

In the present embodiment, solids are stored in the third storage portion 50c so that the solids dispersed after an ejection of liquid may clog the nozzle of an ejection head 42 to inhibit ejection, so as to prevent ejection being performed a plurality of times. Desirably the solids have an average particle diameter larger than the nozzle diameter of the ejection head as well as have specific gravity less than that of the indispensable liquid. Here, the “average particle diameter” of solid particles indicates the most frequently occurring particle diameter channel (mode diameter) in the distribution of sizes of the solid particles. In addition, the nozzle diameter of the ejection head is the diameter of the opening where liquid is ejected as liquid droplets. Specific gravity of the solids less than that of the indispensable liquid is desirable since in such case the solid particles can be guided toward the ejection head to clog the nozzle as liquid is consumed.

To start inhalation of liquid by the inhaler, attachment is performed by inserting the small diameter portion 45a of the reservoir E2 into an applied concave portion 44 of a liquid ejection portion 41 in the direction of an arrow shown in the figure, as shown in FIG. 3A. The following operation is sequentially performed to fill liquid into the ejection head 42 in the period from the beginning to the end of this attachment operation.

The pointed tip of a communicating member 43 breaks through the first film 46 to make the first storage portion 50a communicate with the ejection head 42 so that liquid of a kind other than the indispensable liquid is supplied to the ejection head 42.

Next, as shown in FIG. 3B, the tip of the communicating member 43 breaks through the second film 47 to make the second storage portion 50b communicate with the ejection head 42 so that the indispensable liquid is supplied to the ejection head 42. Next, the tip of the communicating member 43 breaks through the third film 48 to make the third storage portion 50c communicate with the ejection head 42 so that the solids are supplied to the ejection head 42. In other words, a communicating member 43 is provided which makes the first to third storage portions 50a to 50c communicate sequentially with the ejection head 42.

According to this embodiment, the first liquid to be supplied to the ejection head (liquid 51 overflowing from the ejection port) is the liquid not used for inhalation. Therefore in the same way as Embodiment 1, the medicine used for inhalation is not wasted at the times of filling and preliminary ejection. Further, after the medicine used for inhalation is ejected, ejection of liquid droplets is inhibited by the solids being supplied to the ejection head. In this way, the liquid cartridge, which is to be used up by single inhalation, can be prevented from being used repeatedly.

### Embodiment 3

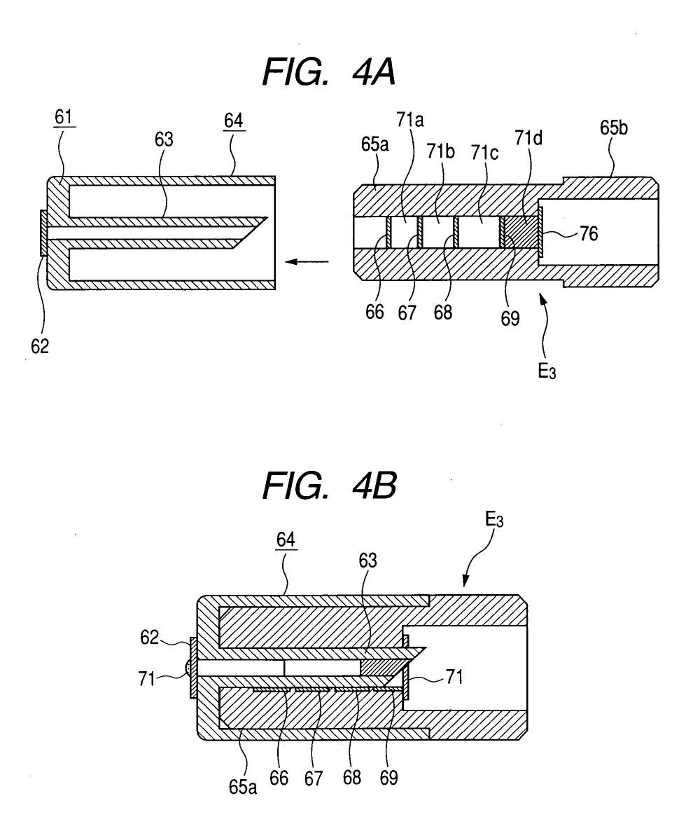

FIGS. 4A and 4B illustrate a liquid cartridge according to Embodiment 3. FIG. 4A is a schematic sectional view of the reservoir and an ejection head 62 before communication. FIG. 4B is a schematic sectional view of the reservoir and the ejection head after communication is completed.

As shown in FIGS. 4A and 4B, a reservoir E3 has a small diameter portion 65a and a large diameter portion 65b of outer and inner diameters slightly larger than those of the small diameter portion 65a. The small diameter portion 65a has a first storage portion 71a to a fourth storage portion 71d isolated by five films 66 to 69, 76 arranged spaced from one another. The first storage portion 71a stores liquid of a kind other than indispensable liquid, and the second storage portion 71b stores an aiding agent for inhalation. The third storage portion 71c stores indispensable liquid and the fourth storage portion 71d stores solids inhibiting ejection.

For a specific example, there is a case where several kinds of medicine are prescribed for bronchial asthma treatment drugs. In addition, a combination is enabled in which the bronchi are dilated by means of a sympathomimetic agent (liquid aiding absorption) to ease breathing and thereafter the bronchi are dilated by means of a parasympatholytic agent (liquid to be ejected) to prevent asthma attacks.

In use for diabetes treatment, first the bronchi are dilated by means of a sympathomimetic agent (liquid aiding absorption) to ease breathing. Thereafter insulin (indispensable liquid) is inhaled to be absorbed from the lung capillary. In this way, inhalation of a plurality of medicines is enabled by a configuration having a plurality of storage portions in which liquid to be inhaled is stored.

The following desirable embodiment can be cited as a summary about a reservoir having a plurality of storage portions isolated by isolation films as in Embodiments 1 to 3.

A plurality of storage portions communicate sequentially with an ejection head in the period from the beginning to the end of attachment operation of a reservoir to an applied portion. Desirably liquid of a kind other than indispensable liquid to be ejected by a liquid ejection device is stored in: the storage portion closest to the ejection head among the plurality of storage portions; that is, a first storage portion which communicates with the ejection head by the attachment operation first.

In addition, desirably solids inhibiting ejection of liquid droplets by the ejection head are stored in: the storage portion most remote from the ejection head among the plurality of storage portions; that is, the storage portion which communicates with the ejection head by the attachment operation last.

### Embodiment 4

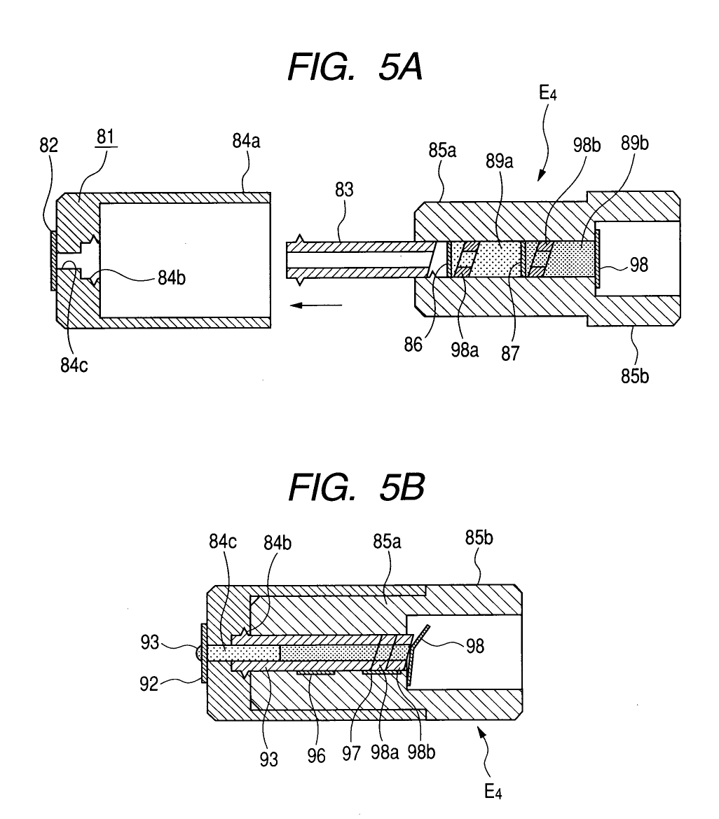

FIGS. 5A and 5B illustrate a liquid cartridge according to Embodiment 4. FIG. 5A is a schematic sectional view before communication is provided between the reservoir and the ejection head. FIG. 5B is a schematic sectional view after communication is provided between the reservoir and the ejection head.

As shown in FIGS. 5A and 5B, a reservoir E4 has a small diameter portion 85a and a large diameter portion 85b of outer and inner diameters slightly larger than those of the small diameter portion 85a. The small diameter portion 85a has a first storage portion 89a and a second storage portion 89b isolated by films 86, 87, 98 which are three isolation films arranged spaced from one another.

The first storage portion 89a stores liquid of a kind other than indispensable liquid to be inhaled, and has a first penetrating member 98a arranged which is movable in the axial direction. The second storage portion 89b has a second penetrating member 98b arranged therein which is movable in the axial direction, as well as the indispensable liquid to be inhaled. A liquid ejection portion 81 has an applied concave portion (applied portion) 84a to which the small diameter portion 85a of the reservoir E4 can be detachably fitted. A fitting concave portion 84b to which the tip of a communicating member 83 is fitted is provided in the central portion of the applied concave portion 84a.

To start inhalation of liquid by the inhaler in the present embodiment, the tip portion of the communicating member 83 is fitted to the fitting portion 84b by pushing the reservoir E4 into the applied concave portion 84a. Thereafter the communicating member 83 breaks through the first film 86 so that the first storage portion 89a and an ejection head 82 communicate and the liquid of a kind other than the indispensable liquid is filled into the liquid ejection portion 81. Desirably a resistance member is installed so that at this time the first film 86 is broken through after the communicating member 83 is fitted to the fitting portion 84b.

The communicating member 83 is further pushed in, thereby to break through the second film 87 through the penetrating member 98a. The communicating member 83 and the two penetrating members 98a, 98b are further pushed in, whereby the liquid of a kind other than the indispensable liquid filled in the liquid ejection portion 81 is pushed out and the indispensable liquid is filled. Thereafter, the second penetrating member 98b breaks through the third film 98 to make an opening communicating with the atmosphere so that liquid overflows at the ejection surface of the ejection head 82.

Since liquid droplets cannot be ejected if liquid remains overflowing at the ejection surface, the surface of the ejection surface is wiped to remove surplus liquid to start ejection.

When a liquid cartridge of the present invention is used by being applied to an inhaler, the indispensable liquid is for example one of a drug solution (medicinal solution such as insulin, vitamin B1), aroma chemical and nicotine which is to be inhaled by the user; the liquid of an other kind is for example one of purified water, saline, alcohol aqueous solution and a solvent for the indispensable liquid.

The reservoir needs to store liquid and maintain the sterilized state, and therefore desirably comprises a material which does not dissolve into the liquid and which is harmless to a living body. For example, polyethylene, flexible polypropylene, polycarbonate, ABS resin, cycloolefin resin and methacryl resin can be cited. In addition, composite resin such as polyethylene/(ethylene vinyl alcohol copolymer) and polypropylene/(ethylene vinyl alcohol copolymer) may be used.

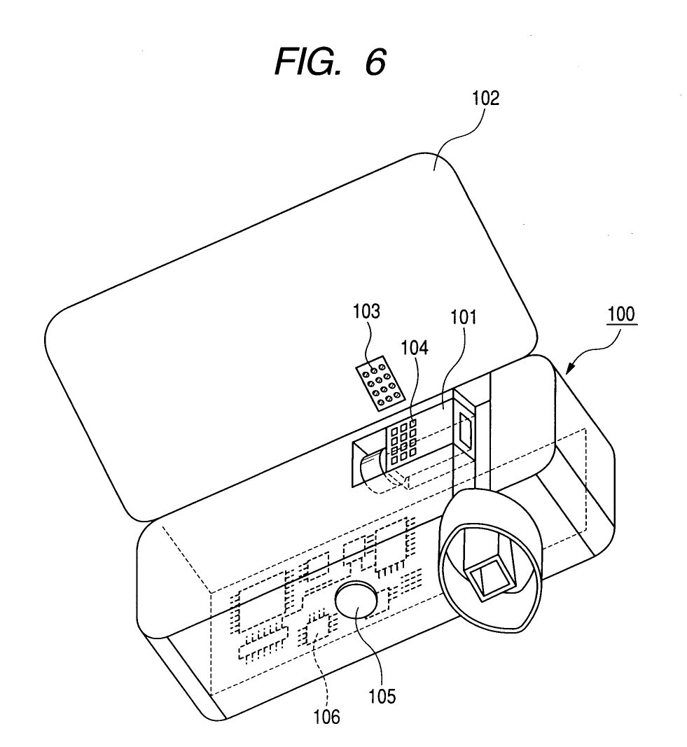

FIG. 6 illustrates a schematic view of an example in which a liquid cartridge of the present invention is applied to an inhaler, the view being a transparent view for part of the main body of the inhaler.

A liquid cartridge 101 is attached to an inhaler 100 after the above-described surplus filling liquid overflowing from the ejection head is removed. Next an access cover 102 of the inhaler 100 is closed. Electrical power is supplied to an electric contact point 104 provided in the liquid cartridge through an inhaler side electric contact point 103 provided in the access cover 102. Predetermined electrical power is supplied for a predetermined time period by pushing an ejection button 105 of the inhaler 100, and the filling liquid and the main purpose liquid are sequentially ejected from the ejection head of the liquid ejection portion. Conditions of atomization such as the predetermined time period are controlled by a controlling unit 106 in the inhaler 100. That is, the controlling unit 106 functions as a control unit for controlling the driving of the ejection head in the side of the liquid cartridge of the present invention. When the nozzle diameter of the ejection head is 1.5 to 3 μm, the ejected liquid droplets will have a particle diameter of about 3 μm and can be delivered into the lungs.

The liquid cartridge of the present invention desirably has information about the volume of the first storage portion (10a, 50a, 71a, 89a) in which a medicine of an other kind is stored. For example, the above-mentioned information may be stored in one of a bar code and an IC tag which is attached to the liquid cartridge. In the inhaler is provided a reading unit which can read the bar code when the liquid cartridge is attached. The controlling unit 106 can drive the ejection head so as to perform preliminary ejection based on the result of reading by the reading unit. For example, when the volume of the first storage portion is 1 mL, 1 mL of the medicine of an other kind is supplied to the ejection head. Considering the amount of liquid which will overflow by the filling action, the ejection head is driven by the controlling unit 106 to perform preliminary ejection such that 0.9 mL of the liquid is ejected. In this way, preliminary ejection can be performed without wasting the medicine to be inhaled. In addition, at the time of inhalation the original medicine to be inhaled can be inhaled from the beginning. Preliminary ejection may be performed automatically by the apparatus immediately before the inhalation after the user has turned on the power to the inhaler and is ready to inhale. Alternatively, preliminary ejection may be performed by the user operating the ejection button 105. The ejection amount of preliminary ejection may be determined in advance by the inhaler side based on the amount of the medicine of an other kind memorized by the liquid cartridge; the mode may also be such that the user inputs a parameter corresponding to the number of times of preliminary ejection, following the above-mentioned information.

The present invention is not limited to the above embodiments and various changes and modifications can be made within the spirit and scope of the present invention. Therefore to apprise the public of the scope of the present invention, the following claims are made.

This application claims the benefit of Japanese Patent Application Nos. 2007-193996, filed Jul. 26, 2007, 2008-163977, filed Jun. 24, 2008, which are hereby incorporated by reference herein in their entirety.

1. A liquid cartridge attachable to a liquid ejection device, comprising:
a reservoir having first and second storage portions isolated by isolation films;
a liquid ejection portion having an ejection head for ejecting liquid droplets and an applied portion to which the reservoir is applied; and
a communicating member for sequentially providing communication of the first and the second storage portions with the ejection head in a period from a beginning to an end of operation of applying the reservoir to the applied portion,
wherein the second storage portion stores indispensable liquid and the first storage portion stores liquid of a kind other than the indispensable liquid.

2. A liquid cartridge attachable to a liquid ejection device, comprising:
a reservoir having first to third storage portions isolated by isolation films;
a liquid ejection portion having an ejection head for ejecting liquid droplets and an applied portion to which the reservoir is applied; and
a communicating member for sequentially providing communication of the first to the third storage portions with the ejection head in a period from a beginning to an end of operation of applying the reservoir to the applied portion,
wherein the second storage portion stores indispensable liquid, the first storage portion stores liquid of a kind other than the indispensable liquid and the third storage portion stores solids for inhibiting ejection of liquid droplets by the ejection head.

3. A liquid cartridge attachable to a liquid ejection device, comprising:
a reservoir having first to fourth storage portions isolated by isolation films;
a liquid ejection portion having an ejection head for ejecting liquid droplets and an applied portion to which the reservoir is applied; and
a communicating member for sequentially providing communication of the first to the fourth storage portions with the ejection head in a period from a beginning to an end of operation of applying the reservoir to the applied portion,
wherein the second and the third storage portions store indispensable liquid, the first storage portion stores liquid of a kind other than the indispensable liquid and the fourth storage portion stores solids for inhibiting ejection of liquid droplets by the ejection head.

4. The liquid cartridge according to claim 1, wherein liquid stored in the first storage portion is a solvent for the indispensable liquid stored in the second storage portion.

5. The liquid cartridge according to claim 1, wherein liquid stored in the first storage portion is any one of purified water, saline and alcohol aqueous solution.

6. The liquid cartridge according to claim 2, wherein the solids have average particle diameter larger than nozzle diameter of the ejection head and have specific gravity less than that of the indispensable liquid.

7. The liquid cartridge according to claim 1, wherein the indispensable liquid is a medicine.

8. A liquid cartridge attachable to a liquid ejection device, comprising:
a reservoir having a plurality of storage portions isolated by isolation films;
a liquid ejection portion having an ejection head for ejecting liquid droplets and an applied portion to which the reservoir is applied; and
a communicating member for sequentially providing communication of the plurality of storage portions with the ejection head in a period from a beginning to an end of operation of applying the reservoir to the applied portion,
wherein a storage portion closest to the ejection head among the plurality of storage portions stores liquid of a kind other than indispensable liquid to be ejected by the liquid ejection device.

9. The liquid cartridge according to claim 8, wherein a storage portion remotest from the ejection head among the plurality of storage portions stores solids for inhibiting ejection of liquid droplets by the ejection head.

10. The liquid cartridge according to claim 1, wherein the liquid cartridge has information about volume of a storage portion in which the liquid of an other kind is stored.

11. An inhaler comprising:
the liquid cartridge according to claim 1; and
a control unit for controlling driving of the ejection head.

12. An inhaler comprising:
the liquid cartridge according to claim 10; and
a reading unit for the information,
wherein the ejection head is driven so as to perform preliminary ejection not inhaled by a user by means of the liquid of an other kind based on a result of reading by the reading unit.
